(19) United States
(12) Patent Application Publication
(10) Pub. No.: US 2008/0188446 A1
(43) Pub. Date: Aug. 7, 2008

(54) TETRACYCLINE COMPOSITIONS FOR TOPICAL ADMINISTRATION

(75) Inventors: Brendan Muldoon, Glengormley (IE); David Woolfson, Belfast (IE); Stephen McCullagh, Belfast (IE)

Correspondence Address: FITZPATRICK CELLA HARPER & SCINTO, 30 ROCKEFELLER PLAZA, NEW YORK, NY, 10112, US

(73) Assignee: WARNER CHILCOTT COMPANY INC., Fajardo, PR (US)

(21) Appl. No.: 12/024,723

(22) Filed: Feb. 1, 2008

Related U.S. Application Data
(60) Provisional application No. 60/887,866, filed on Feb. 2, 2007.

(51) Int. Cl. A61K 31/65

(52) U.S. Cl. 514/154; 514/153

(57) ABSTRACT

Pharmaceutical formulations containing tetracycline for topical administration, as well as methods of making and administering the same, are disclosed.

This application claims the benefit of U.S. Provisional Application No. 60/887,866, filed Feb. 2, 2007.

### BACKGROUND OF THE INVENTION

1. Field of the Invention

This invention relates to tetracycline formulations for topical administration, as well as to methods of making and administering the same.

2. Related Background Art

Topical antibiotics are a widely accepted, effective and well-tolerated treatment for dermatological conditions, including inflammatory acne vulgaris. Topical antibiotics for the treatment of such dermatological conditions offer the advantage of a decreased total absorption of the drug and an accompanying decrease in toxicity, when compared with systemic antibiotics. In addition, topical antibiotics offer the added benefit of applying the antibiotic directly to the targeted lesions.

Topical antibiotics commonly prescribed in the United States are clindamycin and erythromycin. Tetracycline antibiotics are also used in the treatment of dermatological conditions, but topical formulations of tetracycline antibiotics are limited. Meclocycline (an oxytetracycline derivative) has been formulated as a 1% cream (Meclan® or Meclosorb®). Tetracycline hydrochloride (0.22% w/w) is marketed in the United Kingdom under the brand name Topicycline®; this comprises an aqueous ethanol solution of tetracycline hydrochloride in equilibrium with its degradation product, 4-epitetracycline hydrochloride. The product must be reconstituted by mixing tetracycline hydrochloride powder with an aqueous ethanol solution prior to dispensing, whereupon it is only stable for 8 weeks.

Tetracyclines have limited stability in aqueous solutions (A. Kubis et al., “Investigation of stability of tetracycline hydrochloride in methylcellulose gel”, Pharmazie 42:519-520 (1987)). Tetracycline antibiotics are known to be oxidatively unstable and often change from yellow to brown over time (Y. Liang et al., “Stability studies of tetracycline in methanol solution”, J. Chromatography 827:45-55 (1998)). Despite this, efforts have been made in the prior art to formulate tetracycline compositions for topical administration. These efforts have been hindered, however, by the instability of the tetracycline compositions in the presence of water and other protic liquids. As used herein, “protic liquid” refers to any liquid that carries a hydrogen attached to an oxygen (such as in a hydroxyl group), to a nitrogen (such as in an amine group) and further including any molecular liquid which contains dissociable H⁺. In tetracycline formulations in the presence of water and other protic liquids, the tetracyclines typically form various degradation products such as, but not limited to, epitetracycline, anhydrotetracycline, epianhydrotetracycline, which degradation products have negligible antibiotic activity. This leads to a limited, commercially undesirable shelf life for such tetracycline products in aqueous media.

To overcome the stability problem, the tetracycline antibiotics have been incorporated into various non-aqueous vehicles. Solutions of tetracycline antibiotics in alcohol-based solvents are disclosed in, for example, U.S. Pat. Nos. 3,219,529, 3,389,174 and 4,376,118. However, the use of such alcohol-based solvents has not been pharmaceutically acceptable due to the instability of tetracyclines in the presence of water and other protic liquids. The tetracycline antibiotics have also been formulated in nonaqueous ointment bases, which are less desirable in the treatment of acne due to their greasy consistency. This greasiness, in turn, can be associated with poor patient compliance.

U.S. Pat. No. 3,219,529 addresses the problem of stability of tetracycline solutions by including either an ester of a lower polyhydric alcohol with a lower alkyl fatty acid, certain oxygen heterocycles, or amides of higher molecular weight aliphatic fatty acids di-substituted by lower alkyl groups.

U.S. Pat. No. 3,335,055 addresses the stability of tetracycline solutions by including certain magnesium salts.

U.S. Pat. No. 3,389,174 addresses the stability of certain tetracycline solutions by including substantially completely esterified lower alkyl esters of lower aliphatic polyhydroxy alcohols and lower aliphatic acids.

U.S. Pat. No. 3,944,668 addresses the stability of tetracycline solutions in water by compounding it with 8-hydroxyquinoline or a chlorinated and/or alkyl derivative thereof.

U.S. Pat. No. 4,011,313 addresses the stability of tetracycline antibiotics by dispersing or dissolving it in a dialkylated mono- or poly-alkylene glycol vehicle, optionally containing an antioxidant.

U.S. Pat. No. 4,038,388 is directed to a stable topical antimicrobial composition containing tetracycline, oxytetracycline or chlorotetracycline, as well as a specified 8-hydroxy quinoline.

U.S. Pat. No. 4,376,118 concerns a non-aqueous solution of a tetracycline antibiotic salt containing a non-aqueous diluent, a non-aqueous solvent and a non-aqueous non-ionic solubilizer.

Japanese Publication No. 61130228 relates to a solution of minocycline in a polyhydric alcohol such as glycerine and a magnesium compound such as magnesium chloride. The formulation also contains a water-soluble polymer such as carboxymethylcellulose. The formulation is preferably non-aqueous.

Japanese Publication No. 62123120 concerns aminocycline formulation containing a magnesium compound, a polyhydric alcohol, a water-soluble polymer, a specified methacrylate copolymer and a solubilizing agent such as triacetin.

Kubis et al. (Id.) concerns the granulation of tetracycline hydrochloride with ethanol, methanol or acetone. The formulation comprises tetracycline (granulated with ethanol) in a gel containing 0.7% methylcellulose, 5% 1,2-propylene glycol, 5% dimethyl acetamide, and distilled water. Stability data were provided, and the authors conclude that this formulation was stable with respect to viscosity and pH during 3 years at room temperature storage. However, in terms of antibiotic activity, a 10% drop was observed after 2 days at room temperature and after 7 days on storage at 4° C.

U.S. Pat. No. 4,701,320 concerns stabilizing minocycline in a polyhydric alcohol containing a magnesium compound.

U.S. Pat. No. 5,855,904 concerns a method of preparing a sustained release preparation for treating periodontitis, the method comprising forming chitosan-coated microspheres containing an antibiotic selected from tetracycline hydrochloride, tetracycline bases and minocycline hydrochloride.

U.S. Pat. No. 5,122,519 concerns stabilizing certain tetracyclines by avoiding protic solvents and using instead, as a formulation base, a gelling agent comprising polyethylene homopolymer, polyethylene/vinyl acetate copolymer or polyethylene/acrylic acid copolymer, an emollient ester co-solvent and a volatile silicone solvent.

Japanese Patent Publication No. 10114648 describes a topical composition containing a C₂₀₋₂₅ terpene alcohol and an active ingredient such as a tetracycline or benzoyl peroxide for improved percutaneous absorption or chemical stability.

Japanese Patent Publication No. 11286448 describes a topical composition containing minocycline and an aluminum compound in a polyhydric alcohol base.

U.S. Pat. No. 6,566,350 concerns a stable minocycline-containing topical composition. The composition may contain minocycline, an oleaginous base, an adhesive agent and a sucrose fatty acid ester.

U.S. Pat. No. 6,774,100 concerns a composition comprising a polar solvent, such as a polyhydric alcohol, and a thickening agent to achieve a specified viscosity, the thickening agent comprising a polyacrylamide.

U.S. Patent Publication No. US2006/0172982 concerns a topical composition in which a tetracycline is dispersed in a salve selected from petroleum jelly, zinc oxide and boric acid.

U.S. Pat. No. 6,482,810 concerns an antibiotic composition for inhibition of angiogenesis, in which minocycline hydrochloride was incorporated in an ethylene-vinyl acetate copolymer matrix.

International Patent Publication No. 2006/138035 concerns a composition for delivering lipid soluble pharmaceutical agents, the composition comprising a substantially anhydrous mixture containing a solvent, a silicone elastomer, a surfactant and a lipid soluble pharmaceutical agent.

Multiple attempts have been made to devise stabilized tetracycline formulations, as is documented in the patent prior art briefly reviewed above. However, none of the above-mentioned patents or patent applications adequately addresses the stability of a tetracycline antibiotic. Thus, there is a need for a topical tetracycline composition, which is stable and convenient to use, which provides good delivery of the tetracycline to the skin surface, and which is cosmetically acceptable. Accordingly, tetracycline compositions for topical administration, that do not suffer from the deficiencies of conventional topical compositions, are desirable.

### SUMMARY OF THE INVENTION

The present invention is directed to a tetracycline formulation for topical administration comprising at least one tetracycline or a pharmaceutically acceptable salt or hydrate thereof substantially stabilized in a base, wherein the base comprises at least one hydrophobic, non-hygroscopic silicone thickening agent and wherein the formulation is substantially free of protic liquids (including water). In a particularly preferred embodiment, the base further comprises at least one hydrophobic, non-hygroscopic liquid. In a preferred embodiment of the invention, topical administration is external administration to the skin. In certain embodiments, the tetracycline formulation optionally comprises at least one penetration enhancer, at least one preservative, at least one mucoadhesive agent, at least one chelating agent, at least one antioxidant, at least one pharmaceutically acceptable excipient and/or at least one additional pharmaceutically active agent.

Preferred embodiments of the invention include those in which the at least one tetracycline comprises a 1,4,4a,5,5a,6,11,12a-octahydro naphthacene-2-carboxamide structure having two different substituents at one or more of positions 1, 4, 5, 6 and 11, hydrogen being considered a substituent. Preferably the at least one tetracycline has two different substituents at position 4 and, more preferably, the at least one tetracycline has two different substituents at each of positions 4 and 6. Tetracyclines suitable for use in the present invention are those which are unstable in water and other protic liquids. Such tetracyclines include [4S-(4α,4aα,5aα,12aα)]-4-(dimethylamino)-1,4,4a,5,5a,6,11,12a-octahydro-3,10,12,12a-tetrahydroxy-1,11-dioxo-2-naphthacenecarboxamides having two different substituents at one or more of positions 4, 5, and 6. Preferably the at least one tetracycline has two different substituents at position 4 and, more preferably, the at least one tetracycline has two different substituents at each of positions 4 and 6. More preferred embodiments of the invention include those in which the at least one tetracycline has the structural formula:

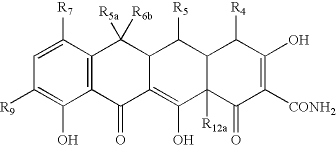

wherein R₄ is selected from the group consisting of a mono(lower alkyl)amino and a di(lower alkyl)amino;
R₉ is selected from the group consisting of hydrogen, a mono(lower alkyl)amino, a di(lower alkyl)amino and 2-(tert-butylamino)acetamido;
R₅ and R₁₂ₐ are independently selected from the group consisting of hydrogen and hydroxyl;
R₆ₐ and R_{6b} are independently selected from the group consisting of hydrogen, lower alkyl and hydroxyl, or can together form ═CH₂;
R₇ is selected from the group consisting of hydrogen, a halogen such as chloride, a mono(lower alkyl)amino and a di(lower alkyl)amino;
or a pharmaceutically acceptable salt or hydrate thereof. In a more preferred embodiment, the at least one tetracycline is selected from the group consisting of doxycycline and minocycline and their pharmaceutically acceptable salts or hydrates. In a still more preferred embodiment, the at least one tetracycline is minocycline or a pharmaceutically acceptable salt or hydrate thereof.

In an optional embodiment of the invention, the at least one hydrophobic, non-hygroscopic liquid is a hydrophobic, non-hygroscopic silicone liquid. In a preferred embodiment of such a formulation, at least one penetration enhancer is also present. In a further optional embodiment of the invention, the tetracycline formulation comprises at least one tetracycline or a pharmaceutically acceptable salt or hydrate thereof substantially stabilized in a base, wherein the base consists essentially of at least one hydrophobic, non-hygroscopic silicone liquid; at least one hydrophobic, non-hygroscopic silicone thickening agent and at least one penetration enhancer, and wherein the formulation is substantially free of protic liquids.

The present invention is also directed to a method of making a tetracycline formulation comprising the step of mixing at least one tetracycline or a pharmaceutically acceptable salt or hydrate thereof in a base, wherein the base comprises at least one hydrophobic, non-hygroscopic silicone thickening agent and wherein the formulation is substantially free of protic liquids. In a particularly preferred embodiment, the base further comprises at least one hydrophobic, non-hygroscopic liquid. The present invention is further directed to tetracycline formulations made according to the methods of the invention.

The present invention is still further directed to a method of treating a dermatological condition comprising the step of administering a tetracycline formulation to an accessible body surface of a human or an animal in need of such treatment, wherein the tetracycline formulation comprises at least one tetracycline or a pharmaceutically acceptable salt or hydrate thereof substantially stabilized in a base, wherein the base comprises at least one hydrophobic, non-hygroscopic silicone thickening agent and wherein the formulation is substantially free of protic liquids. In a particularly preferred embodiment, the base further comprises at least one hydrophobic, non-hygroscopic liquid. In a preferred embodiment of the present invention, the method further comprises an optional step of mixing the tetracycline formulation with at least one protic liquid to render the at least one tetracycline suitable for topical administration. The protic liquid may be mixed with the tetracycline formulation before, after or simultaneously upon administration of the tetracycline formulation to an accessible body surface.

### DETAILED DESCRIPTION OF THE INVENTION

The present invention is directed to a tetracycline formulation for topical administration comprising at least one tetracycline or a pharmaceutically acceptable salt or hydrate thereof substantially stabilized in a base. The base comprises at least one hydrophobic, non-hygroscopic silicone thickening agent and the formulation is substantially free of protic liquids. In a particularly preferred embodiment of the invention, the base further comprises at least one hydrophobic, non-hygroscopic liquid. Preferably the formulation of the invention is suitable for external administration to the skin.

The tetracycline formulation contains at least one tetracycline or a pharmaceutically acceptable salt or hydrate thereof substantially stabilized in a base. As used herein, at least one tetracycline that is “substantially stabilized” in a formulation refers to a formulation in which preferably more than about 85%, and more preferably more than about 90%, of the at least one tetracycline or its pharmaceutically acceptable salt or hydrate remains after storage at 25° C. and 60% relative humidity (RH) for preferably about 3 months, more preferably about 6 months, and still more preferably about 12 months. “Substantially stabilized” can also refer to a formulation in which preferably more than about 85%, and more preferably more than about 90%, of the at least one tetracycline or its pharmaceutically acceptable salt or hydrate retains its antibiotic activity after storage at 25° C. and 60% relative humidity for preferably about 3 months, more preferably about 6 months, and still more preferably about 12 months. In a preferred embodiment of the invention, the at least one tetracycline is substantially suspended in the base. As used herein, “substantially suspended” means preferably at least about 50%, more preferably at least about 75%, still more preferably at least about 85%, and most preferably at least about 95%, of the at least one tetracycline or its pharmaceutically acceptable salt or hydrate is suspended in the base at 32° C. In these preferred embodiments of the invention, in which the at least one tetracycline is substantially suspended in the base, the formulation is substantially free of any surfactant. As used herein, “substantially free” refers to the presence of preferably less than about 1%, more preferably less than about 0.75%, still more preferably less than about 0.5%, still further more preferably less than about 0.1%, and most preferably less than about 0.01%, w/w surfactants. Without being bound by theory, it is thought that the at least one tetracycline can be substantially stabilized by being substantially suspended and, therefore, physically separated from those agents that cause a reduction in antibiotic activity. For this reason, in the preferred embodiments of the invention, the use of surfactant is undesirable, the aim, instead, being to maintain the at least one tetracycline substantially in suspension.

“Tetracycline” refers to a number of antibiotics derived from a system of four linearly annelated six-membered rings (1,4,4a,5,5a,6,11,12a-octahydronaphthacene) with a characteristic arrangement of double bonds. Certain known tetracyclines comprise 1,4,4a,5,5a,6,11,12a-octahydro naphthacene-2-carboxamide structures. Tetracyclines suitable for use in the present invention are those which are unstable in water and other protic liquids. Such tetracyclines include a 1,4,4a,5,5a,6,11,12a-octahydro naphthacene-2-carboxamide structure having two different substituents at one or more of positions 1, 4, 5, 6 and 11, hydrogen being considered a substituent. Preferably the at least one tetracycline has two different substituents at position 4 and, more preferably, the at least one tetracycline has two different substituents at each of positions 4 and 6. Tetracyclines suitable for use in the present invention are those which are unstable in water and other protic liquids. Such tetracyclines include [4S-(4α,4aα,5aα,12aα)]-4-(dimethylamino)-1,4,4a,5,5a,6,11,12a-octahydro-3,10,12,12a-tetrahydroxy-1,11-dioxo-2-naphthacenecarboxamides having two different substituents at one or more of positions 4, 5, and 6. Preferably the at least one tetracycline has two different substituents at position 4 and, more preferably, the at least one tetracycline has two different substituents at each of positions 4 and 6. More preferably such tetracyclines include, without limitation, those having the following structural formula:

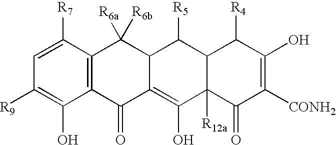

in which R₄ is selected from the group consisting of a mono(lower alkyl)amino and a di(lower alkyl)amino;
R₉ is selected from the group consisting of hydrogen, a mono(lower alkyl)amino, a di(lower alkyl)amino and 2-(tert-butylamino)acetamido;
R₅ and R₁₂ₐ are independently selected from the group consisting of hydrogen and hydroxyl;
R₆ₐ and R_{6b} are independently selected from the group consisting of hydrogen, lower alkyl and hydroxyl, or can together form ═CH₂;
R₇ is selected from the group consisting of hydrogen, a halogen such as chloride, a mono(lower alkyl)amino and a di(lower alkyl)amino;
or a pharmaceutically acceptable salt or hydrate thereof. The presence of such a mono- or di-(lower alkyl)amino substituent at R₄ is believed to render a tetracycline unstable to water and other protic liquids—the 4-epimer degradation product has negligible antibacterial activity. The 6-epimer can form when the R₆ₐ and R_{6b} substituents are different. Tetracyclines suitable for use in this invention also include pharmaceutically acceptable salts and hydrates of suitable tetracyclines, in particular, but not limited to, non-toxic acid addition salts such as hydrochloric, sulfonic and trichloroacetic acid salts. Tetracyclines suitable for use in the present invention also include prodrugs and derivatives thereof, provided they share the naphthacene core structure and include at least one substituent that is unstable to water and other protic liquids.

Exemplary tetracyclines represented by the above structural formula include, without limitation, tetracycline; 7-methylamino-6-deoxy-6-demethyltetracycline; 7-ethylamino-6-deoxy-6-demethyltetracycline; 7-isopropylamino-6-deoxy-6-demethyltetracycline; 9-methylamino-6-deoxy-6-demethyltetracycline; 9-ethylamino-6-deoxy-6-demethyltetracycline; 9-isopropylamino-6-deoxy-6-demethyltetracycline; 7,9-di(ethylamino)-6-deoxy-6-demethyltetracycline; 7-dimethylamino-6-deoxy-6-demethyltetracycline (minocycline); 9-dimethylamino-6-deoxy-6-demethyltetracycline; 7-methylamino-6-deoxytetracycline; 9-ethylamino-6-deoxytetracycline; 7,9-di(methylamino)-6-deoxytetracycline; 7-diethylamino-6-deoxytetracycline; 9-diethylamino-6-deoxytetracycline; 7,9-di(methylethylamino)-6-deoxytetracycline; 7-methylamino-9-ethylamino-6-deoxytetracycline; 9-methylamino-5-hydroxy-6-deoxytetracycline; 6-deoxy-5-hydroxytetracycline (doxycycline); oxytetracycline; 7-chlorotetracycline; 7-chloro-6-demethyltetracycline; 6-methyleneoxytetracycline; tigecycline and the pharmaceutically acceptable salts and hydrates of the foregoing.

More preferred tetracyclines include, without limitation, tetracycline; 7-dimethylamino-6-deoxy-6-demethyltetracycline; 7-methylamino-6-deoxy-6-demethyltetracycline; 9-methylamino-6-deoxy-6-demethyltetracycline; 7-ethylamino-6-deoxy-6-demethyltetracycline; 7-isopropylamino-6-deoxy-6-demethyltetracycline; 6-deoxy-5-hydroxytetracycline; oxytetracycline; 7-chlorotetracycline; 7-chloro-6-demethyltetracycline; 6-methyleneoxytetracycline; tigecycline and the pharmaceutically acceptable salts and hydrates of the foregoing. Specific examples of the most preferred tetracyclines include, without limitation, tetracycline, minocycline, doxycycline, oxytetracycline, chlortetracycline, demeclocycline, methacycline, tigecycline, and the pharmaceutically acceptable salts or hydrates of the foregoing. Special mention is made of minocycline and doxycycline, and their pharmaceutically acceptable salts or hydrates. Minocycline and its salts and hydrates are especially preferred for use in the present invention. Minocycline is a potent semi-synthetic tetracycline with activity against a wide range of gram-positive and gram-negative organisms. It has been shown to be particularly effective as adjunctive therapy in the treatment of severe acne.

According to the present invention, the at least one tetracycline is preferably employed in an amount ranging from about 0.00001% to about 10%, more preferably in an amount ranging from about 0.0025% to about 6%, and most preferably in an amount ranging from about 0.01% to about 3%, by weight of the tetracycline formulation.

Bases suitable for use are hydrophobic and non-hygroscopic. As used herein, “non-hygroscopic” refers to a material which does not readily take up water. As used herein, “hydrophobic” refers to being non-polar and thus having no affinity for water. Preferred hydrophobic and non-hygroscopic bases (and their individual constituents) can have a contact angle of greater than about 90 degrees. Bases suitable for use comprise silicone thickening agents or combinations of silicone thickening agents with liquid vehicles. In one preferred embodiment, the base comprises at least one hydrophobic, non-hygroscopic silicone thickening agent. In another preferred embodiment, the base further comprises at least one hydrophobic, non-hygroscopic liquid.

Hydrophobic, non-hygroscopic silicone thickening agents are suitable for use as the base in the present invention. It is to be understood that at least one hydrophobic, non-hygroscopic silicone thickening agent is required, but that combinations of more than one hydrophobic, non-hygroscopic silicone thickening agents are contemplated. Silicone thickening agents partly or wholly comprise one or more polysiloxane-derived components. A polysiloxane-derived component is defined as any constituent comprising the general chemical motif —[Si(R¹)(R²)—O]ₙ—, in which n defines the number of repeat units (chemical motifs) in the polysiloxane and may take values in the range from about 5 to about 1,000,000; in which part or all of the backbone of the polysiloxane-derived component comprises alternating silicon (S) and oxygen (O) atoms; and in which R¹ and R² groups, which may be the same or different, are selected from a wide range of chemical ligands known in the art. Examples include, but are not limited to, alkyl, vinyl, hydrogen, aryl and fluoride ligands. Preferably, the R¹ and R² groups, which are the same or different, are alkyl groups such that the silicone thickening agent is nominally derived from polydialkylsiloxane, and most preferably the R¹ and R² groups are each methyl ligands, such that the silicone thickening agent is nominally derived from polydimethylsiloxane. Optionally, the silicone thickening agent(s) or combinations thereof may be chemically crosslinked according to methods known by those skilled in the art. Alternatively, the silicone thickening agent may be an amino-functional silicone. Such silicones are cationic silicones with an enhanced ability to bind to keratinaceous substrates. Further alternatively, the silicone thickening agent may be an anionic silicone.

If the formulation is in the form of a gel, paste or ointment, the at least one hydrophobic, non-hygroscopic silicone thickening agent comprises at least 5% w/w, optionally greater than 7.5% w/w, and more optionally between 7.5 and 15% w/w, of the overall formulation. If the formulation is in the form of a lotion, the at least one hydrophobic, non-hygroscopic silicone thickening agent comprises at least 0.5% w/w, optionally at least 1% w/w, and further optionally at least 2% w/w of the overall formulation.

In a preferred embodiment, the hydrophobic, non-hygroscopic silicone thickening agent is preferably a silicone elastomer, and combinations thereof, wherein the at least one polysiloxane-derived component is physically or chemically crosslinked to form a three-dimensional polymeric network. Silicone elastomers can be prepared by a crosslinking reaction between (A) ≡Si—H containing polysiloxanes and (B) an alpha, omega-diene in the presence of a platinum catalyst and (C) a low molecular weight linear or cyclic polysiloxane, as described in U.S. Pat. No. 5,654,362. The elastomers can be swollen with a low molecular weight polysiloxane under a shear force.

Particularly preferred silicone thickening agents include the chemically crosslinked ST-Elastomer 9041 (a silicone elastomer in dodecamethyl pentasiloxane) and ST-Elastomer 10 (also known as Dow Corning 9040 Silicone Elastomer Blend; which is a mixture of high molecular weight silicone elastomer (12% w/w) in decamethylcyclopentasiloxane) in which the mixture has been prior thickened and does not require post-shearing. If the formulation is in the form of a gel, paste or ointment, the at least one silicone elastomer comprises at least 5% w/w, optionally greater than 7.5% w/w, and more optionally between 7.5 and 15% w/w, of the overall formulation. If the formulation is in the form of a lotion, the at least one silicone elastomer comprises at least 0.5% w/w, optionally at least 1% w/w, and further optionally at least 2% w/w of the overall formulation.

Suitable silicone elastomers can be prepared as described in U.S. Pat. No. 5,654,362 and International Patent Publication No. WO 2006/138035, the disclosures of which are incorporated by reference in their entirety. More specifically, silicone oils or other solvents can be thickened to a gel-like consistency by reacting (A) a ≡Si—H containing polysiloxane of formula R₃SiO(R′₂SiO)ₐ(R″HSiO)_{b}SiR₃ and optionally a ≡Si—H containing polysiloxane of formula HR₂SiO(R′₂SiO)_{c}SiR₂H or formula HR₂SiO(R′₂SiO)ₐ(R″HSiO)_{b}SiR₂H where R, R′, and R″ are alkyl groups with 1-6 carbon atoms; a is 0-250; b is 1-250; and c is 0-250; with (B) an alpha, omega-diene of formula CH₂═CH(CH₂)ₓCH═CH₂ where x is 1-20. The reaction is conducted in the presence of a platinum catalyst, in the presence of (C) a low molecular weight silicone oil or other solvent. The reaction is continued until a gel is formed by crosslinking and addition of ≡Si—H across double bonds in the alpha, omega-diene.

The ≡Si—H containing polysiloxane (A) is represented by compounds of the formula R₃SiO(R′₂SiO)ₐ(R″HSiO)_{b}SiR₃ designated as type A¹ and compounds of the formula HR₂SiO(R′₂SiO)_{c}SiR₂H or formula HR₂SiO(R′₂SiO)ₐ(R″HSiO)_{b}SiR₂H designated h as type A². In these formulae, R, R′, and R″, are alkyl groups with 1-6 carbon atoms; a is 0-250; b is 1-250; and c is 0-250. The reaction can be conducted using only compounds of type A¹. If both types A¹ and A² are present, the molar ratio of compounds A²:A¹ is 0-20, preferably 0-5.

The alpha, omega-diene (B) is a compound of the formula CH₂═CH(CH₂)ₓ CH═CH₂ where x is 1-20. Some representative examples of suitable alpha, omega-dienes for use herein are 1,4-pentadiene; 1,5-hexadiene; 1,6-heptadiene; 1,7-octadiene; 1,8-nonadiene; 1,9-decadiene; 1,11-dodecadiene; 1,13-tetradecadiene; and 1,19-eicosadiene.

The addition and crosslinking reaction requires a catalyst to effect the reaction between the ≡SiH containing polysiloxane and the alpha, omega-diene. Suitable catalysts are Group VIII transition metals, i.e., the noble metals. Such noble metal catalysts are described in U.S. Pat. No. 3,923,705, incorporated herein by reference to show platinum catalysts. One platinum catalyst is Karstedt's catalyst, which is described in Karstedt's U.S. Pat. Nos. 3,715,334 and 3,814,730, incorporated herein by reference. Karstedt's catalyst is a platinum divinyl tetramethyl disiloxane complex typically containing about one weight percent of platinum in a solvent such as toluene. Another platinum catalyst is a reaction product of chloroplatinic acid and an organosilicon compound containing terminal aliphatic unsaturation and is described in U.S. Pat. No. 3,419,593, incorporated herein by reference. The noble metal catalysts are used in amounts from 0.00001-0.5 parts per 100 weight parts of the ≡SiH containing polysiloxane, preferably 0.00001-0.02 parts, most preferably 0.00001-0.002 parts.

The phrase low molecular weight silicone oil (C) includes (i) low molecular weight linear and cyclic volatile methyl siloxanes, (ii) low molecular weight linear and cyclic volatile and non-volatile alkyl and aryl siloxanes, and (iii) low molecular weight linear and cyclic functionalized siloxanes; these materials are described below with regard to silicone liquids suitable for optional use in the base of the tetracycline formulation of the invention.

Other suitable silicone thickening agents comprise copolymers comprising a polysiloxane (including, but not limited to, a polydimethylsiloxane) and an ester, an amide or an ether, including, but not limited to, polyoxyalkylene ether.

Still further suitable silicone thickening agents comprise graft copolymers comprising a polysiloxane (including, but not limited to, a polydimethylsiloxane) and polyvinyls, polyethylene, polypropylene, polystyrene, polyacrylates and polyurethanes.

Silicone-based thickening agents seem to be associated with improved skin feel. It is postulated that silicones provide a silky skin feel, by reducing tack and improving spreading, but without greasiness. Without being bound by theory, it is expected that the improved skin feel and the decreased perception of greasiness should improve user compliance.

In a particularly preferred embodiment of the present invention, the base further comprises a hydrophobic, non-hygroscopic liquid. Again, it is to be understood that at least one hydrophobic, non-hygroscopic liquid is required, but that combinations of more than one hydrophobic, non-hygroscopic liquids are contemplated.

Suitable hydrophobic, non-hygroscopic liquid vehicles include, without limitation, mineral oils, silicone liquids, non-protic liquids such as, without limitation, decylmethyl sulfoxide and dialkyl isosorbides such as dimethyl isosorbide, and combinations thereof. As used herein, “non-protic liquids” refer to liquids that share ion dissolving power with protic liquids but which lack the dissociable H⁺, otherwise known as the acidic hydrogen of a polar liquid. In contrast, protic liquids do have such a dissociable H⁺, for example, a hydrogen attached to an oxygen (such as in a hydroxyl group) or to a nitrogen (such as in an amine group).

Suitable silicone liquids include, without limitation, linear and cyclic siloxane polymers and copolymers, for example, alkyl, haloalkyl and aryl, linear and cyclic, siloxane polymers and copolymers. For example, suitable silicone liquids include, without limitation, linear and cyclic alkyl and aryl siloxanes such as linear polydimethylsiloxane (commonly known as silicone oil), cyclopolydimethylsiloxanes (cyclomethicones) including, but not limited to, decamethylcyclopentasiloxane, further including, without limitation, low molecular weight linear and cyclic volatile methyl siloxanes; low molecular weight linear and cyclic volatile and non-volatile alkyl and aryl siloxanes; and low molecular weight linear and cyclic functionalized siloxanes. Also included within the scope of functionalized silicone liquids are halosilicone liquids, including fluorosilicone liquids, further including, without limitation, trifluoropropylmethyl siloxane. Also included within the scope of silicone liquids are copolymers thereof, including, without limitation, dimethylsiloxane and trifluoropropylmethylsiloxane copolymers supplied by, for example, Nusil. Further included within the scope of functionalized silicone liquids are hydride- and vinyl-functionalized silicone liquids, including, without limitation, hydride- and vinyl-functionalized linear and cyclic alkyl, haloalkyl and aryl siloxane polymers and copolymers.

Low molecular weight linear and cyclic volatile methyl siloxanes (VMS) are considered suitable silicone liquids. VMS compounds correspond to the average unit formula (CH₃)ₐSiO₍₄₋ₐ₎/2 in which a has an average value of two to three. The compounds contain siloxane units joined by ≡Si—O—Si≡ bonds. Representative units are monofunctional “M” units (CH₃)₃SiO₁/2 and difunctional “D” units (CH₃)₂SiO₂/2. The presence of trifunctional “T” units CH₃SiO₃/2 results in the formation of branched linear or cyclic volatile methyl siloxanes. The presence of tetrafunctional “Q” units SiO₄/2 results in the formation of branched linear or cyclic volatile methyl siloxanes.

Linear VMS have the formula (CH₃)₃SiO{(CH₃)₂SiO}_{y}Si(CH₃)₃. The value of y is 0-5. Cyclic VMS have the formula {(CH₃)₂SiO}_{z}. The value of z is 3-6. Preferably, these volatile methyl siloxanes have boiling points less than about 250° C. and viscosities of about 0.65-5.0 centistokes (mm²/s). Representative linear volatile methyl siloxanes are hexamethyldisiloxane (MM) with a boiling point of 100° C., viscosity of 0.65 mm²/s, and formula Me₃ SiOSiMe₃; octamethyltrisiloxane (MDM) with a boiling point of 152° C., viscosity of 1.04 mm²/s, and formula Me₃SiOMe₂SiOSiMe₃; decamethyltetrasiloxane (MD₂M) with a boiling point of 194° C., viscosity of 1.53 mm²/s, and formula Me₃SiO(Me₂SiO)₂SiMe₃; dodecamethylpentasiloxane (MD₃M) with a boiling point of 229° C., viscosity of 2.06 mm²/s, and formula Me₃SiO(Me₂SiO)₃SiMe₃; tetradecamethylhexasiloxane (MD₄M) with a boiling point of 245° C., viscosity of 2.63 mm²/s, and formula Me₃SiO(Me₂SiO)₄SiMe₃; and hexadecamethylheptasiloxane (MD₅M) with a boiling point of 270° C., viscosity of 3.24 mm²/s, and formula Me₃SiO(Me₂SiO)₅SiMe₃. Representative cyclic volatile methyl siloxanes are hexamethylcyclotrisiloxane (D₃) a solid with a boiling point of 134° C. and formula {(Me₂)_{SiO}}₃; octamethylcyclotetrasiloxane (D₄) with a boiling point of 176° C., viscosity of 2.3 mm²/s, and formula {(Me₂)_{SiO}}₄; decamethylcyclopentasiloxane (D₅) with a boiling point of 210° C., viscosity of 3.87 mm²/s, and formula {(Me₂)_{SiO}}₅; and dodecamethylcyclohexasiloxane (D₆) with a boiling point of 245° C., viscosity of 6.62 mm²/s, and formula {(Me₂)_{SiO}}₆. Representative branched volatile methyl siloxanes and are heptamethyl-3-{(trimethylsilyl)oxy}trisiloxane (M₃T) with a boiling point of 192° C., viscosity of 1.57 mm²/s, and formula C₁₀H₃₀O₃Si₄; hexamethyl-3,3,bis{(trimethylsilyl)oxy}trisiloxane (M₄Q) with a boiling point of 222° C., viscosity of 2.86 mm²/s, and formula C₁₂H₃₆O₄Si₅; and pentamethyl {(trimethylsilyl)oxy}cyclotrisiloxane (MD₃) with the formula C₈H₂₄O₄Si₄. Low molecular weight linear and cyclic volatile and non-volatile alkyl and aryl siloxane can also be used. Representative linear polysiloxanes are compounds of the formula R₃SiO(R₂SiO)_{y}SiR₃, and representative cyclic polysiloxanes are compounds of the formula (R₂SiO)_{z}. R is an alkyl group of 1-6 carbon atoms, or an aryl group such as phenyl. The value of y is 0-80, optionally 0-20. The value of z is 0-9, optionally 4-6. These polysiloxanes have viscosities generally in the range of about 1-100 centistokes (mm²/s).

The base, and indeed, the entire formulation, must be substantially free of protic liquids, such as water and as defined above. As used herein, “substantially free” refers to the presence of preferably less than about 10.0%, more preferably less than about 5.0%, still more preferably less than about 2.5%, and most preferably less than about 0.75%, w/w protic liquids. “Substantially free” can also refer to the presence of preferably less than about 0.75% w/w, and more preferably less than about 0.50%, w/w free water. As used herein, “free water” refers to water not associated with the tetracycline or its pharmaceutically acceptable salt or hydrate. Examples of protic liquids include, but are not limited to, water, alcohols such as methanol, ethanol, glycerol, polyhydric alcohols and glycols such as ethylene glycol, propylene glycol and polyethylene glycol, acids such as acetic acid and formic acid, and bases such as ammonia.

The base may further include one or more optional ingredients such as mucoadhesive agents, penetration enhancers, pharmaceutically acceptable excipients, antioxidants, chelating agents, additional pharmaceutically active agents and preservatives. When present, such optional ingredients are included in an amount, which can be readily determined by one of ordinary skill in the art. Furthermore, one of ordinary skill in the art would readily appreciate that care should be taken in selecting optional ingredients (mucadhesive agents, penetration enhancers, pharmaceutically acceptable excipients, antioxidants, chelating agents, additional pharmaceutically active agents and preservatives) so as not to include an ingredient in the base which would compromise the substantial stability of the at least one tetracycline therein.

The tetracycline composition of the present invention may contain at least one mucoadhesive agent. As used herein, “mucoadhesive” refers to adhering to a biological substrate comprising mucosal surfaces. Suitable mucoadhesive agents include, without limitation, the copolymers of poly(methylvinylether/maleic anhydride), known commercially as Gantrez copolymers, in order to enhance the residence time of the final composition on the mucosal surface at the site of application.

Still another optional ingredient is at least one penetration enhancer. As used herein, “penetration enhancer” refers to an agent that alters the movement of the active ingredient across the skin, either by a direct interaction on the skin or by adjusting the physico-chemical characteristics of the active ingredient or both. Penetration enhancers suitable for use in the present invention include, without limitation, azone, dimethyl sulfoxide, oleic acid, d-limonene, or a fatty acid ester optionally formed from a fatty acid comprising from 2 to 20 carbon atoms (such as, but not limited to caproic acid, lauric acid, myristic acid, oleic acid, linoleic acid, adipic acid and lanolic acid) optionally esterified with an alcohol of 2 to 20 carbon atoms, such as an alkanol of 2 to 4 carbon atoms, menthol and the non-ionic alkoxylates (such as, but not limited to, Arlamol). Fatty acid esters are preferred penetration enhancers. A particularly preferred fatty acid ester penetration enhancer for inclusion in the base is isopropyl myristate. In a preferred embodiment of this invention, the base consists essentially of at least one hydrophobic, non-hygroscopic silicone thickening agent, at least one hydrophobic, non-hygroscopic liquid and a penetration enhancer.

Another optional ingredient is at least one antioxidant and/or at least one chelating agent. Suitable antioxidants and chelating agents useful in the context of the present invention include, but are not limited to, ascorbic acid and its salts, citric acid and its salts, edatate and its salts and tocopherol and its derivatives.

Still another optional ingredient is at least one additional pharmaceutically acceptable excipient. Suitable excipients include, without limitation, waxes (such as white soft paraffin) and, if the topical composition is a foam, suitable propellants such as liquefied propellants (for example, propane, isobutene, n-butane, dimethyl ether and the chlorofluorocarbons) and combinations thereof.

Another optional ingredient is at least one preservative. Suitable preservatives include, without limitation, para-hydroxybenzoate derivatives commonly known as parabens.

At least one additional pharmaceutically active agent may also be optionally included in the tetracycline formulation of the present invention. In one preferred embodiment, however, the at least one tetracycline is the only pharmaceutically active agent present. As used herein, “pharmaceutically active agent” or “agent” or “drug” or “active agent” or “active ingredient”, etc., refers to any agent capable of defending against, or treating, a disease or cosmetic state (infection control or skin disease) in the human or animal body, or a prodrug thereof. Such pharmaceutically active agents may be organic or inorganic and may be prophylactically or therapeutically active, systemically or locally. Alternatively or additionally, such pharmaceutically active agents may be cosmetically active. As used herein, “prophylactically active” refers to an agent's (or its prodrug's) effectiveness in defending against a disease state in the human or animal body, preferably the human body. As used herein, “therapeutically active” refers to an agent's (or its prodrug's) effectiveness in treating a disease state in the human or animal body, preferably the human body. As used herein, “cosmetically active” refers to an agent's (or its prodrug's) effectiveness in defending against or treating a cosmetic condition in or on the human or animal body, preferably the human body. Typically, the additional pharmaceutically active agent is selected from anti-inflammatory compounds (such as diclofenac, ibuprofen, ketoprofen), antimicrobials (such as clindamycin and erythromycin) and the like, keratolytic agents such as benzoyl peroxide, azelic acid, retinoids, calcineurin antagonists, immunomodulators, and combinations thereof. The term “retinoids” includes first generation retinoids such as retinol, tretinoin, isotretinoin and alitretinoin, second generation retinoids such as etretinate and its metabolite, acitretin, and third generation retinoids such as tazarotene and bexarotene.

The tetracycline formulation may take the form of a semi-solid preparation (such as a gel, paste or ointment), a pourable preparation (such as a lotion), or a foam. The final form requires that the tetracycline be substantially stabilized in the base; in addition, the final form should optionally allow for rendering the at least one tetracycline suitable for topical administration upon mixture of the formulation with an external source of protic liquid. It should be apparent to one of ordinary skill in the art that the final form will be dependent upon the composition of the base, i.e., presence and amounts of viscosity enhancers, solvents, etc.

As used herein, “semi-solid” is understood to refer to the rheological properties of the formulations themselves, such that the formulations will flow under an applied force but will remain in situ following application to any accessible body surface. As used herein, a “lotion” is a dermatological vehicle that is a pourable suspension of insoluble powder in a liquid. As used herein, a “gel” is a semi-solid vehicle that consists of a liquid phase that is constrained within a three-dimensional polymeric network. The polymeric network may be formed by chemical (covalent crosslinks) or physical (hydrogen bonds, Van der Waals forces) interactions between polymer chains (more correctly, between functional groups on polymer chains). Where the liquid phase is non-aqueous, the gel is an organogel. Oleogels are lipophilic gels whose bases typically consist of liquid paraffin with polyethylene or fatty oils gelled with colloidal silica or a long-chain fatty acid soap. As used herein, an “ointment” base is a semi-solid vehicle composed of hydrophobic constituents. Ointments can take the form of non-hydrocarbon ointment. Ointments related to the present invention can be formulated to provide a non-greasy, cosmetically acceptable appearance. As used herein, a “paste” is an ointment with a high loading of insoluble solids (up to 50% by weight) that forms a structured particulate matrix. As used herein, a “foam” is a disperse system consisting of a three dimensional network of films in air. Foams have a high surface area and tend to spontaneous collapse unless stabilized.

Upon optional mixture of the tetracycline formulation of the present invention with an external source of protic liquid, the tetracycline is rendered suitable for topical delivery. As used herein, “rendered suitable for topical delivery” or “rendered suitable for topical administration” refers to the availability of tetracycline to be absorbed by an accessible body surface and present in an amount effective to topically treat a disease condition such as acne or rosacea. It is desired that the tetracycline be in a molecularly dispersed form to facilitate topical delivery. In particularly preferred embodiments of the present invention, the tetracycline which is substantially stabilized in the base is substantially solubilized by the external source of protic liquid, upon mixture. As used herein, “substantially solubilized” refers to preferably at least about 50%, more preferably at least about 75%, still more preferably at least about 85%, and most preferably at least about 95%, of the at least one tetracycline or its salt or hydrate is solubilized at 32° C. in the topical composition, upon mixture of the formulation with the protic liquid.

Alternatively, the tetracycline formulation is suitable for topical administration without the need for an external source of protic liquid. In this alternative embodiment, it is postulated that acne is associated with increased sebum production. At least some of the tetracycline might dissolve into either sebum or perspiration and, thereby, be carried onto, or into, an accessible body surface, such as the skin.

The exceptional stability of the tetracycline in the base eliminates the need for any reconstitution from a powder (i.e., there is no dry powder component in the present invention) prior to dispensing and saves the patient expense because there is no need for special storage or frequent replacement.

The present invention is further directed to a method of making a tetracycline formulation comprising the step of mixing at least one tetracycline or a pharmaceutically acceptable salt or hydrate thereof in a base, wherein the base comprises at least one hydrophobic, non-hygroscopic silicone thickening agent and wherein the formulation is substantially free of protic liquids. In a particularly preferred embodiment, the base further comprises at least one hydrophobic, non-hygroscopic liquid. All of the details regarding the tetracycline, the bases, the components, etc. are the same as set forth above with regard to the first embodiment of the invention. Preparation of the formulation can be accomplished by any suitable method using any suitable means, e.g., by admixture of the ingredients typically through the use of vigorous agitation such as high shear mixing. Optional additional steps include those which result in the addition of one or more of the optional ingredients set forth above with respect to the first embodiment.

The present invention is still further directed to a tetracycline formulation made according to the methods of the second embodiment of the invention.

Another embodiment of the invention is directed to a method of treating a dermatological condition comprising the step of administering a tetracycline formulation to an accessible body surface of a human or an animal in need of such treatment, wherein the tetracycline formulation comprises at least one tetracycline or a pharmaceutically acceptable salt or hydrate thereof substantially stabilized in a base, wherein the base comprises at least one hydrophobic, non-hygroscopic silicone thickening agent and wherein the formulation is substantially free of protic liquids. In a particularly preferred embodiment, the base further comprises at least one hydrophobic, non-hygroscopic liquid. As used herein, “topical administration” refers to administration onto any accessible body surface of any human or animal species, preferably the human species, for example, the skin or mucosal epithelia. In certain embodiments of this invention, “topical” refers to an external application to the skin epithelium.

An optional step of the present inventive method comprises mixing the tetracycline formulation with a suitable volume of at least one protic liquid such as to render the at least one tetracycline suitable for topical administration. One of ordinary skill in the art can readily determine a suitable volume. The protic liquid should have a pH ranging preferably from about 4 to about 8 and is most preferably water or primarily water. Mixing together of the formulation and the at least one protic liquid can be accomplished by any suitable method using any suitable manual or automated means.

If the tetracycline formulation is to be mixed with a suitable volume of at least one protic liquid, the at least one protic liquid is supplied independently or via an external source, i.e., not included in the tetracycline formulation. The method of this embodiment contemplates mixing of the formulation and the at least one protic liquid prior to administration to the accessible body surface, after administration to the accessible body surface or at the time of administration to the accessible body surface. In other words, the formulation and the protic liquid may be applied to the accessible body surface of the human or animal as a mixture together or they may separately be applied to the accessible body surface of the human or animal, in either order. What is more, when applied together, the formulation and the protic liquid may be mixed together at the time of application to the body surface or they may be mixed prior to the application to the body surface. The at least one protic liquid may be supplied to an accessible body surface; for example, the protic liquid can be supplied by providing an appropriate amount of protic liquid for mixture (before, during or after administration of the tetracycline formulation to the accessible body surface), by splashing water on the accessible body surface to which the tetracycline formulation will be applied, by washing without drying the accessible body surface to which the tetracycline formulation will be applied, etc. Alternatively, the at least one protic liquid may already be present on an accessible body surface at the time of application of the tetracycline formulation due to perspiration or some other natural moisture of the accessible body surface. When the tetracycline formulation and the protic liquid are mixed prior to application, one of ordinary skill in the art will recognize that mixture should be accomplished just prior to application.

As used herein, “dermatological condition” refers to cosmetic and pathological disorders of the skin. Dermatological conditions include topical inflammatory skin conditions such as eczema, contact dermatitis, rosacea, psoriasis and acne including acne rosacea. As used herein, “acne” is a disorder of the skin characterized by papules, pustules, cysts, nodules, comedones, and other blemishes or skin lesions. These blemishes and lesions are often accompanied by inflammation of the skin glands and pilosebaceous follicles, as well as, microbial, especially bacterial, infection. For the purposes of this specification, acne includes all known types of acne. Some types of acne include, for example, acne vulgaris, cystic acne, acne atrophica, bromide acne, chlorine acne, acne conglobata, acne cosmetica, acne detergicans, epidemic acne, acne estivalis, acne fulminans, halogen acne, acne indurata, iodide acne, acne keloid, acne mechanica, acne papulosa, pomade acne, premenstral acne, acne pustulosa, acne scorbutica, acne scrofulosorum, acne urticata, acne varioliformis, acne venenata, propionic acne, acne excoriee, gram negative acne, steroid acne, nodulocystic acne and acne rosacea. Acne rosacea is characterized by inflammatory lesions (erythema) and telangiectasia. Telangiectasia is abnormally and permanently dilated blood vessels associated with a number of diseases. For example, facial telangiectasia is associated with age, acne rosacea, sun exposure, and alcohol use. The present invention can also be used to treat certain other types of acneiform dermal disorders, e.g. perioral dermatitis, seborrheic dermatitis in the presence of acne, gram negative folliculitis, sebaceous gland dysfunction, hiddradenitis suppurativa, pseudo-folliculitis barbae, or folliculitis.

Specific embodiments of the invention will now be demonstrated by reference to the following general methods of manufacture and examples. It should be understood that these examples are disclosed solely by way of illustrating the invention and should not be taken in any way to limit the scope of the present invention.

### EXAMPLE 1

A tetracycline formulation for topical administration was prepared using the ingredients set forth in Table 1 below.

TABLE 1

| Ingredient | % w/w |
|---|---|
| ST-Elastomer 10 | 75.00 |
| ST-Cyclomethicone - NF | to 100.00 |
| Isopropyl Myristate | 10.00 |
| Minocycline HCl* | 1.42 |

*1.20% w/w minocycline free base

First, the cyclomethicone and the minocycline HCl were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

### EXAMPLE 2

A tetracycline formulation for topical administration was prepared using the ingredients set forth in Table 2 below.

TABLE 2

| Ingredient | % w/w |
|---|---|
| Doxycycline Hyclate* | 0.231 |
| ST-Elastomer 10 | 80.000 |
| ST-Cyclomethicone-5-NF | to 100.000 |
| Isopropyl Myristate | 1.000 |

*0.20% w/w doxycycline free base

First, the cyclomethicone and the doxycycline hyclate were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

### EXAMPLE 3

A tetracycline formulation for topical administration was prepared using the ingredients set forth in Table 3 below.

TABLE 3

| Ingredient | % w/w |
|---|---|
| Doxycycline Monohydrate* | 0.208 |
| ST-Elastomer 10 | 80.000 |
| ST-Cyclomethicone-5-NF | to 100.000 |
| Isopropyl Myristate | 1.000 |

*0.20% w/w doxycycline free base

First, the cyclomethicone and the doxycycline monohydrate were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

### EXAMPLE 4

A tetracycline formulation for topical administration was prepared using the ingredients set forth in Table 4 below.

TABLE 4

| Ingredient | % w/w |
|---|---|
| Minocycline HCl* | 0.237 |
| Cyclomethicone | to 100.000 |
| ST-Elastomer 10 | 75.000 |
| Isopropyl myristate | 1.000 |

*0.20% w/w minocycline free base

First, the cyclomethicone and the minocycline HCl were mixed in a beaker, following which the isopropyl myristate was then added, to form a mixture. Next, the mixture was added to the ST-Elastomer 10 with stirring at room temperature (about 20° C.). The stirring continued until the mixture was substantially mixed with the ST-Elastomer 10.

### EXAMPLE 5

A tetracycline formulation for topical administration was prepared using the ingredients set forth in Table 5 below.

TABLE 5

| Ingredient | % w/w |
|---|---|
| Minocycline HCl* | 0.473 |
| ST-Elastomer 10 | 80.00 |
| ST-Cyclomethicone-5-NF | to 100.00 |
| Isopropyl Myristate | 1.00 |

*0.40% w/w minocycline free base

First, the cyclomethicone and the minocycline HCl were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

### EXAMPLE 6

A tetracycline formulation for topical administration was prepared using the ingredients set forth in Table 6 below.

TABLE 6

| Ingredient | % w/w |
|---|---|
| Minocycline HCl* | 2.37 |
| ST-Elastomer 10 | 75.00 |
| ST-Cyclomethicone-5-NF | to 100.00 |
| Isopropyl Myristate | 1.00 |

*2.00% w/w minocycline free base

First, the cyclomethicone and the minocycline HCl were mixed in a beaker, following which the isopropyl myristate was added, to form a mixture. Next, the mixture was added to the ST-Elastomer 10 with stirring at room temperature (about 20° C.). The stirring continued until the mixture was substantially mixed with the ST-Elastomer 10.

### EXAMPLE 7

A tetracycline formulation for topical administration was prepared using the ingredients set forth in Table 7 below.

TABLE 7

| Ingredient | % w/w |
|---|---|
| Minocycline HCl* | 0.473 |
| ST-Elastomer 10 | 75.00 |
| ST-Cyclomethicone-5-NF | to 100.00 |

*0.40% w/w minocycline free base

First, the cyclomethicone and the minocycline HCl were mixed in a beaker to form a mixture. Next, the mixture was added to the ST-Elastomer 10 with stirring at room temperature (about 20° C.). The stirring continued until the mixture was substantially mixed with the ST-Elastomer 10.

### EXAMPLE 8

A tetracycline formulation for topical administration is prepared using the ingredients set forth in Table 8 below.

TABLE 8

| Ingredient | % w/w |
|---|---|
| Minocycline HCl* | 1.89 |
| ST-Elastomer 10 | 75.00 |
| Cyclomethicone-5 NF | to 100.00 |
| Isopropyl Myristate | 10.00 |

*1.60% w/w minocycline free base

First, the cyclomethicone and the minocycline HCl are mixed in a beaker, following which the isopropyl myristate is added, to form a mixture. Next, the mixture is added to the ST-Elastomer 10 with stirring at room temperature (about 20° C.). The stirring continues until the mixture is substantially mixed with the ST-Elastomer 10.

### EXAMPLE 9

A tetracycline formulation for topical administration was prepared using the ingredients set forth in Table 9 below.

TABLE 9

| Ingredient | % w/w |
|---|---|
| Minocycline HCl* | 1.89 |
| Cyclomethicone | to 100.000 |
| ST-Elastomer 10 | 75.000 |

*1.60% w/w minocycline free base

First, the cyclomethicone and the minocycline HCl were mixed in a beaker to form a mixture. Next, the mixture was added to the ST-Elastomer 10 with stirring at room temperature (about 20° C.). The stirring continued until the mixture was substantially mixed with the ST-Elastomer 10.

### EXAMPLE 10

A tetracycline formulation for topical administration is prepared using the ingredients set forth in Table 10 below.

TABLE 10

| Ingredient | % w/w |
|---|---|
| Caprylic/capric triglyceride | 24.50 |
| Mineral oil | 10.00 |
| Cyclomethicone | 32.00 |
| Beeswax | 1.50 |
| Sorbitan monooleate | 6.00 |
| ST-Elastomer 10 | 25.00 |
| Minocycline HCl* | 1.00 |

*0.85% w/w minocycline free base

All ingredients except the minocycline HCl are weighed in a vessel and heated to 70-75° C. with mixing until a uniform consistency is produced. The mixture is then cooled to 35° C. before gradually adding the minocycline HCl. The minocycline HCl-containing mixture (active mixture) is then cooled to ambient temperature and added to an aerosol compartment of a foam canister. The compartment is sealed and appropriate amount of propellant (5-25% w/w of the composition mass) is pressurized in an aluminum aerosol can with hydrofluorocarbon.

The formulation of Table 10 and the separate external source of protic liquid are mixed together, either upon expulsion from the foam canister or before or during application to the skin. Alternatively, the formulation of Table 10 and the separate second external source of protic liquid are mixed together upon expulsion from either the foam canister or are applied from two sources after sequential application, in either order, to the skin.

### EXAMPLE 11

A tetracycline formulation for topical administration was prepared using the ingredients set forth in Table 11 below.

TABLE 11

| Ingredient | % w/w |
|---|---|
| Minocycline HCl* | 0.59 |
| Cyclomethicone | to 100.000 |
| ST-Elastomer 10 | 75.000 |
| Isopropyl myristate | 1.000 |

*0.5% w/w minocycline free base

First, the cyclomethicone and the minocycline HCl were mixed in a beaker, following which the isopropyl myristate was then added, to form a mixture. Next, the mixture was added to the ST-Elastomer 10 with stirring at room temperature (about 20° C.). The stirring continued until the mixture was substantially mixed with the ST-Elastomer 10.

### Stability Testing

### Storage Stability of Doxycycline Hyclate Gel

The tetracycline formulation of Example 2 was tested for stability. Specifically, aliquots of the composition were stored in aluminum tubes for up to 3 months at 25° C. and 60% RH and for up to 4.5 months at 40° C. and 75% RH. The doxycycline content was assessed by HPLC after storage. To do this, first the doxycycline was extracted from the formulation by taking a sample using a displacement pipette and pipetting directly into a tared volumetric flask. Absolute ethanol (30 ml) was then added, and the sample was then sonicated for 2-3 minutes. Deionized water was then used to accurately adjust to the required volume of 50 ml in the volumetric flask. Finally, the sample was filtered through a 0.45 μm filter prior to injection onto the HPLC column. The HPLC parameters used were as follows:

| | |
|---|---|
| Analytical Column: | Packing Type = Gemini RP18 |
| | Particle Size = 5 μm |
| | Column Length = 250 mm |
| | Internal Diameter = 4.6 mm |
| Mobile Phase (doxycycline): | 60 volumes KH₂PO₄ buffer (0.1M) |
| | 40 volumes methanol (HPLC) |
| | Adjust to pH 7.8 using 5N NaOH |
| | Add 0.5 g tetra-butyl ammonium hydrogen |
| | sulphate per litre after adjusting pH. |
| Flow rate: | 1.0 mL/min |
| Column Temperature: | 45° C. |
| Injection Volume: | 10 μL |
| Detection Wavelength: | 270 nm |

The results of the storage stability are shown in Tables 12 and 13 below.

TABLE 12
Tetracycline Formulation of Example 2 stored at 25° C./60% RH.

| Active % Recovery | | % Area 4-Epimer | | % Area 6-Epimer | |
|---|---|---|---|---|---|
| 0 month | 3 months | 0 month | 3 months | 0 month | 3 months |
| 100.8 | 101.5 | ND | ND | 0.45 | 0.46 |

TABLE 13
Tetracycline Formulation of Example 2 stored at 40° C./75% RH.

| | | | | |
|---|---|---|---|---|
| 0 month | 1 month | 2 months | 3 months | 4.5 months |

Active % Recovery*

| | | | | |
|---|---|---|---|---|
| 100.8 | 103.3 | 103.4 | 99.1 | 105.4 |

% Area 4-Epimer

| | | | | |
|---|---|---|---|---|
| ND | ND | ND | ND | ND |

% Area 6-Epimer

| | | | | |
|---|---|---|---|---|
| 0.45 | 0.42 | 0.43 | 0.42 | 0.47 |

ND means not detected
*numbers are mean value for 2 samples

As can be seen, after 3 months of storage at 25° C. and 60% relative humidity, the amount of doxycycline recovered remained unchanged. After 4.5 months of storage at 40° C. and 75% relative humidity, the amount of doxycycline recovered remained substantially unchanged. After 3 months, the amount of the 4-epimer (a degradation product, 4-epidoxycycline) is undetectable both at 40° C. and 75% RH and at 25° C. and 60% RH. The amount of the 6-epimer (a degradation product, 6-epidoxycycline) did not change over this time period both at 40° C. and 75% RH and at 25° C. and 60% RH.

### Storage Stability of Doxycycline Monohydrate Gel

The tetracycline formulation of Example 3 was tested for stability. Specifically, aliquots of the composition were stored in aluminum tubes for 4.5 months at 40° C. and 75% RH. The doxycycline content was assessed by HPLC after storage in a manner similar to that described above. The results of the storage stability are shown in Table 14 below.

TABLE 14
Tetracycline Formulation of Example 3 stored at 40° C./75% RH.

| | | | | |
|---|---|---|---|---|
| 0 month | 1 month | 2 months | 3 months | 4.5 months |

Active % Recovery*

| | | | | |
|---|---|---|---|---|
| 104.9 | 98.2 | 107.5 | 102.1 | 103.3 |

% Area 4-Epimer

| | | | | |
|---|---|---|---|---|
| ND | ND | ND | ND | ND |

% Area 6-Epimer

| | | | | |
|---|---|---|---|---|
| 0.32 | 0.32 | 0.30 | 0.31 | 0.37 |

ND means not detected
*numbers are mean value for 2 samples

As can be seen, after 4.5 months of storage at 40° C. and 75% relative humidity, the amount of doxycycline recovered remained substantially unchanged. After 4.5 months, the amount of the 4-epimer (4-epidoxycycline) is undetectable at 40° C./75% RH. The amount of the 6-epimer (6-epidoxycycline) did not change over this time period at 40° C./75% RH.

### Storage Stability of Minocycline HCl Gel

The formulation of Example 4 was tested for stability. Specifically, aliquots of the composition were stored in aluminum tubes for up to 6 months at 25° C. and 60% RH and for up to 6 months at 40° C. and 75% relative humidity. The minocycline content was assessed by HPLC after storage. To do this, first the minocycline was extracted from the formulation by taking a sample using a displacement pipette and pipetting directly into a tared volumetric flask. Absolute ethanol (30 ml) was then added, and the sample was then sonicated for 2-3 minutes. Deionized water was then used to accurately adjust to the required volume of 50 ml in the volumetric flask. Finally, the sample was filtered through a 0.45 μm filter prior to injection onto the HPLC column. The limit of detection was 0.02 μg/ml (minocycline HCl). The mobile phase for the HPLC method consisted of methanol and acetonitrile. The HPLC parameters used were as follows:

| | |
|---|---|
| Analytical Column: | Packing Type = Gemini RP18 |
| | Particle Size = 5 μm |
| | Column Length = 250 mm |
| | Internal Diameter = 4.6 mm |
| Flow rate: | 1.0 mL/min |
| Column Temperature: | 45° C. |
| Injection Volume: | 10 μL |
| Detection Wavelength: | 270 nm |

A minocycline primary standard was made up by accurately weighing about 55 mg of minocycline HCl reference material into a 100 mL volumetric flask; dissolving and making up to volume with water, stoppering and mixing well. From this primary standard, a secondary standard was made up by accurately transferring 5.0 mL of the minocycline primary standard into a 100 mL volumetric flask and making up to volume with water. When required, aminocycline tertiary standard was made up by accurately transferring 5.0 mL of the minocycline primary standard into a 250 mL volumetric flask and making up to volume with water. The amount of minocycline was calculated using the formula below:

$$Sample Conc / µg \text{/} ml = \frac{{S}_{w} \times P}{100} \times \frac{5}{100} \times \frac{5}{250} \times \frac{{A}_{sample}}{{A}_{std}} \times 1000$$

where:
S_{w}=Standard amount in mg
P=Decimal purity of standard as minocycline
Aₛₐₘₚₗₑ=Area of sample peak
A_{std}=Area of standard peak

The results of the storage stability are shown below in Tables 15 and 16.

TABLE 15
Tetracycline Formulation of Example 4 stored at 25° C./60% RH.

| Active % Recovery | | | % Area 4-Epimer | | |
|---|---|---|---|---|---|
| 0 month | 3 months | 6 months | 0 month | 3 months | 6 months |
| 92.7 | 91.2 | 95.1 | 1.6 | 1.6 | 1.7 |

TABLE 16
Tetracycline Formulation of Example 4 stored at 40° C./75% RH.

| | | | | |
|---|---|---|---|---|
| 0 month | 1 month | 2 months | 4.5 months | 6 months |

Active % Recovery

| | | | | |
|---|---|---|---|---|
| 86.2 | 89.4 | 87.0 | 98.2 | 89.2 |

% Area 4-Epimer

| | | | | |
|---|---|---|---|---|
| 1.6 | 1.4 | 1.7 | 1.6 | 1.7 |

The amount of the minocycline recovered after 6 months storage at 25° C./60% RH and 40° C./75% RH did not differ significantly from the starting level. The amount of the 4-epimer (4-epiminocycline) in the samples stored at 25° C./60% RH and 40° C./75% RH did not change over 6 months storage.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims.

What is claimed is:

1. A tetracycline formulation for topical administration comprising at least one tetracycline or a pharmaceutically acceptable salt or hydrate thereof substantially stabilized in a base,
wherein the base comprises at least one hydrophobic, non-hygroscopic silicone thickening agent, and
wherein the formulation is substantially free of protic liquids.

2. The tetracycline formulation of claim 1, wherein the base further comprises at least one hydrophobic, non-hygroscopic liquid.

3. The tetracycline formulation of claim 1, wherein topical administration is external administration to the skin.

4. The tetracycline formulation of claim 1, wherein more than about 85% of the at least one tetracycline or its pharmaceutically acceptable salt or hydrate remains after storage at 25° C. and 60% relative humidity for 3 months.

5. The tetracycline formulation of claim 1, wherein the at least one tetracycline is substantially suspended in the base.

6. The tetracycline formulation of claim 1, wherein the at least one hydrophobic, non-hygroscopic silicone thickening agent is selected from the group consisting of polysiloxanes and combinations thereof.

7. The tetracycline formulation of claim 1, wherein the at least one hydrophobic, non-hygroscopic silicone thickening agent is a silicone elastomer.

8. The tetracycline formulation of claim 2, wherein the at least one hydrophobic, non-hygroscopic liquid is selected from the group consisting of mineral oils, silicone liquids, non-protic liquids and combinations thereof.

9. The tetracycline formulation of claim 8, wherein the at least one hydrophobic, non-hygroscopic liquid is a silicone liquid.

10. The tetracycline formulation of claim 1, wherein the at least one tetracycline comprises a [4S-(4α,4aα,5aα,12aα)]-4-(dimethylamino)-1,4,4a,5,5a,6,11,12a-octahydro-3,10,12,12a-tetrahydroxy-1,11-dioxo-2-naphthacenecarboxamide having two different substituents at one or more of positions 4, 5 and 6.

11. The tetracycline formulation of claim 10, wherein the at least one tetracycline has the structural formula: 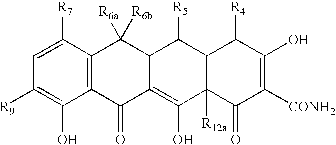
wherein R₄ is selected from the group consisting of a mono(lower alkyl)amino and a di(lower alkyl)amino;
R₉ is selected from the group consisting of hydrogen, a mono(lower alkyl)amino, a di(lower alkyl)amino and 2-(tert-butylamino)acetamido;
R₅ and R₁₂ₐ are independently selected from the group consisting of hydrogen and hydroxyl;
R₆ₐ and R_{6b} are independently selected from the group consisting of hydrogen, lower alkyl and hydroxyl, or can together form ═CH₂;
R₇ is selected from the group consisting of hydrogen, a halogen such as chloride, a mono(lower alkyl)amino and a di(lower alkyl)amino;
or a pharmaceutically acceptable salt or hydrate thereof.

12. The tetracycline formulation of claim 11, wherein the at least one tetracycline is selected from the group consisting of tetracycline; 7-methylamino-6-deoxy-6-demethyltetracycline; 7-ethylamino-6-deoxy-6-demethyltetracycline; 7-isopropylamino-6-deoxy-6-demethyltetracycline; 9-methylamino-6-deoxy-6-demethyltetracycline; 9-ethylamino-6-deoxy-6-demethyltetracycline; 9-isopropylamino-6-deoxy-6-demethyltetracycline; 7,9-di(ethylamino)-6-deoxy-6-demethyltetracycline; 7-dimethylamino-6-deoxy-6-demethyltetracycline; 9-dimethylamino-6-deoxy-6-demethyltetracycline; 7-methylamino-6-deoxytetracycline; 9-ethylamino-6-deoxytetracycline; 7,9-di(methylamino)-6-deoxytetracycline; 7-diethylamino-6-deoxytetracycline; 9-diethylamino-6-deoxytetracycline; 7,9-di(methylethylamino)-6-deoxytetracycline; 7-methylamino-9-ethylamino-6-deoxytetracycline; 9-methylamino-5-hydroxy-6-deoxytetracycline; 6-deoxy-5-hydroxytetracycline; oxytetracycline; 7-chlorotetracycline; 7-chloro-6-demethyltetracycline; 6-methyleneoxytetracycline; tigecycline and the pharmaceutically acceptable salts and hydrates.

13. The tetracycline formulation of claim 12, wherein the at least one tetracycline is selected from the group consisting of minocycline, doxycycline and the pharmaceutically acceptable salts and hydrates of minocycline and doxycycline.

14. The tetracycline formulation of claim 1, wherein the at least one tetracycline is employed in an amount ranging from about 0.00001% to about 10% by weight of the tetracycline formulation.

15. The tetracycline formulation of claim 1 further comprising at least one optional ingredient selected from the group consisting of mucoadhesive agents, penetration enhancers, antioxidants, chelating agents, additional pharmaceutically active agents, preservatives and pharmaceutically acceptable excipients.

16. The tetracycline formulation of claim 15, wherein the additional pharmaceutically active agent is selected from the group consisting of anti-inflammatory compounds, antimicrobials, benzoyl peroxide, azelic acid, retinoids, immunomodulators, and calcineurin antagonists.

17. A method of making a tetracycline formulation comprising the step of mixing at least one tetracycline or a pharmaceutically acceptable salt or hydrate thereof in a base,
wherein the base comprises at least one hydrophobic, non-hygroscopic silicone thickening agent, and
wherein the formulation is substantially free of protic liquids.

18. The method of claim 17, wherein the base further comprises at least one hydrophobic, non-hygroscopic liquid.

19. A tetracycline formulation made according to the method of claim 17.

20. A tetracycline formulation made according to the method of claim 18.

21. A method of treating a dermatological condition comprising the step of:
administering a tetracycline formulation to an accessible body surface of a human or an animal in need of such treatment,
wherein the tetracycline formulation comprises at least one tetracycline or a pharmaceutically acceptable salt or hydrate thereof substantially stabilized in a base,
wherein the base comprises at least one hydrophobic, non-hygroscopic silicone thickening agent, and
wherein the formulation is substantially free of protic liquids.

22. The method of claim 21, wherein the base further comprises at least one hydrophobic, non-hygroscopic liquid.

23. The method of claim 21, wherein the topical administration is external administration to the skin.

24. The method of claim 21 further comprising the step of mixing the tetracycline formulation with at least one protic liquid to render the at least one tetracycline suitable for topical administration.

25. The method of claim 24, wherein the at least one protic liquid is mixed with the tetracycline formulation prior to administration to the accessible body surface.

26. The method of claim 24, wherein the at least one protic liquid is mixed with the tetracycline formulation after administration to the accessible body surface.

27. The method of claim 24, wherein the at least one protic liquid is mixed with the tetracycline formulation at the time of administration to the accessible body surface.

28. The method of claims 24, wherein the at least one protic liquid is provided by an external source of protic liquid.

29. The method of claim 24, wherein the at least one protic liquid is water.
